(19) United States
(12) Patent Application Publication
(10) Pub. No.: US 2024/0325136 A1
(43) Pub. Date: Oct. 3, 2024

(54) OPERATION DEVICE AND METHOD OF OPERATING MEDICAL CARE SYSTEM

(71) Applicant: TERUMO KABUSHIKI KAISHA, Tokyo (JP)

(72) Inventors: Kazuaki KANAMOTO, Hadano city (JP); Yoshiki MISHIMA, Cupertino, CA (US); Yoichiro Kuwano, Tokyo (JP)

(73) Assignee: TERUMO KABUSHIKI KAISHA, Tokyo (JP)

(21) Appl. No.: 18/616,407

(22) Filed: Mar. 26, 2024

(30) Foreign Application Priority Data
Mar. 27, 2023 (JP) 2023-050105

(51) Int. Cl. A61F 2/01; A61B 17/3207; A61B 34/00; A61B 90/00

(52) U.S. Cl. CPC A61F 2/013; A61B 17/320725; A61B 34/71; A61F 2/0105; A61F 2/011; A61B 2090/035

(57) ABSTRACT

An operation device configured to be joined with a proximal portion of a treatment device having a lumen through which a wire extending from an indwelling unit indwelling in a living body lumen to the outside of the body, and includes: a housing including a joining portion to which the treatment device can be joined; and an action unit that is disposed on the proximal side of the joining portion, and the action unit is brought into contact with the wire extending from the treatment device, to apply, to the wire, a moving force for the wire to move in the longitudinal axis direction.

### CROSS-REFERENCES TO RELATED APPLICATIONS

This application claims priority to Japanese Application No. 2023-050105 filed on Mar. 27, 2023, the entire content of which is incorporated herein by reference.

### TECHNOLOGICAL FIELD

The present disclosure generally relates to an operation device for operating a treatment device inserted into a living body lumen, and a method of operating a medical care system.

### BACKGROUND DISCUSSION

As a method of treating an intravenous thrombus, there is a method by which a catheter is percutaneously inserted into a vein to physically crush and remove the thrombus (see, for example, U.S. Pat. No. 8,435,228). In such treatment, pulmonary emboli may appear when thrombi fully or partially detached from the blood vessel wall ride in the bloodstream and reach the lungs. To avoid such pulmonary emboli, there is a known method by which a device in which a filter for collecting thrombi is installed at the distal portion of a wire extending in a longitudinal length is placed in a blood vessel before thrombus crushing. By this method, a thrombus crushing device is inserted into a blood vessel along an elongated wire extending from the filter placed in a blood vessel to the outside of the body.

However, the elongated wire extending from the filter to the outside of the body may unintentionally move due to a sliding force received from the thrombus crushing device or the like that is inserted and removed via the wire. When the proximal portion of the wire moves, the position of the filter placed in the body might be displaced accordingly, or the shape of the filter developed to cover the blood vessel lumen might be canceled.

Also, the mesh portion of the filter is subjected to a flow force, and can easily move to the heart side. When collecting thrombi in the mesh portion, the filter is subjected to a greater flow force than when thrombi are not being collected. Since the filter is secured to the blood vessel wall by its own radial expansion force, the force of securing the filter to the blood vessel wall becomes weaker, and the filter can easily move to the heart side, in a case where the mesh portion of the filter is placed in a large blood vessel. For example, assuming that the inferior vena cava is the indwelling position of the filter, the blood vessel diameter on the heart side might be larger, and the filter is likely to move to the heart side. In some cases, the filter fits into the renal vein to create a gap between the mesh portion and the blood vessel wall, making it difficult to collect thrombi.

Therefore, it is desirable to secure the filter to the blood vessel wall, and for this purpose, it is preferable to press the wire extending from the filter to the outside of the body with a hand or the like. However, when the thrombus crushing device is inserted or removed along the wire by the over-the-wire (OTW) technique, a suction device is pushed or pulled to suction the crushed thrombi, or the thrombus crushing device is connected to a drive unit for driving the thrombus crushing device, movement of the wire due to temporary release of the wire extending from the filter or movement of the wire due to a sliding force might occur, and unintended displacement of the filter might occur. In particular, when the thrombus crushing device is inserted and removed along the wire, unintended displacement of the filter or loosening of the wire might occur due to slidability between the wire and the thrombus crushing device. Also, movement of the filter due to a force received from the blood flow might occur.

### SUMMARY

The present disclosure provides a wire position adjustment device capable of adjusting movement of a wire.

(1) An operation device according to the present disclosure is an operation device configured to be joined with a proximal portion of an elongated treatment device having a lumen through which a wire extending from an indwelling unit indwelling in a living body lumen, the operation device includes: a housing including a joining portion configured to be joined to the proximal portion of the treatment device; and an action unit that is disposed on the proximal side of the joining portion, and wherein the action unit is configured to brought into contact with the wire extending from the proximal portion of the treatment device, to apply, to the wire, a moving force for the wire to move in a longitudinal axis direction.

The operation device of (1) can move the wire in the longitudinal axis direction with the action unit, and thus, it is possible to prevent the indwelling unit from unintentionally moving with respect to the living body lumen, and the wire from unintentionally loosening. Thus, when the treatment device is used, the operation device can maintain appropriate positions and shapes of the indwelling unit joined to the wire extending through the lumen of the treatment device, and the wire.

(2) In the operation device of (1), the action unit may include at least one rotating roller having an outer peripheral surface configured to come into contact with the wire. With this arrangement, the operation device can reliably and highly accurately apply a moving force to the wire through the rotating rollers. As the rotating rollers can be arranged in a space-saving manner, the operation device can be prevented from becoming relatively larger in size.

(3) In the operation device of (2), at least part of the outer peripheral surface of the rotating roller may be formed with a rubber material. With this arrangement, the rotating rollers are less likely to slip even on the wire having a thin and slippery surface, and a force can be reliably applied to the wire.

(4) The operation device of (2) or (3) may further include a limiting mechanism configured to limit rotation of the rotating roller. With this arrangement, the operation device can limit rotation of the rotating rollers holding the wire, to restrict the wire from unintentionally moving with respect to the housing. Thus, the operation device can help prevent the indwelling unit in a living body lumen from unintentionally moving upon receipt of a body fluid, for example, by limiting movement of the wire with the limiting mechanism.

(5) The operation device of any one of (1) to (4) may further include a movement detection unit configured to detect a movement amount in the longitudinal axis direction and a movement direction of the housing. With this arrangement, in the operation device, the movement direction and the movement amount of the wire that are caused by movement of the housing in the action unit necessary for maintaining the appropriate positions and shapes of the indwelling unit and the wire can be specified by the movement detection unit.

(6) In the operation device of (5), the movement detection unit may include at least one detecting roller configured to rotate with respect to the housing, and at least part of the outer peripheral surface of the detecting roller is located outside the housing. With this arrangement, when the operator wishes to move the housing, the detecting rollers are brought into contact with the contact target object such as the patient, a surgical table, or an object placed on the surgical table, to move the housing. Thus, the movement amount and the movement direction of the housing can be reliably and highly accurately detected.

(7) The operation device of (5) or (6) may further include a transmission unit configured to actuate the action unit to move the wire in a direction opposite to the movement direction by a distance corresponding to the movement amount detected by the movement detection unit. With this arrangement, the operation device can move the wire in the direction opposite to the movement of the housing. Thus, it is possible to automatically help prevent the wire from moving with movement of the housing and the indwelling unit from unintentionally moving with respect to a living body lumen, and prevent the wire from unintentionally loosening. Thus, when the treatment device is used, the operation device can effectively maintain appropriate positions and shapes of the indwelling unit joined to the wire extending through the lumen of the treatment device, and the wire.

(8) In the operation device of (7), the transmission unit may include at least one gear configured to transmit rotation detected by the detecting roller to the action unit. With this arrangement, the operation device can reliably and highly accurately transmit rotation detected by the movement detection unit to the action unit.

(9) The operation device according to any one of (1) to (8) may further include a wire holding unit that is disposed outside the housing and is configured to hold the wire, and the wire holding unit is configured to rotate about the longitudinal axis direction while limiting movement of the wire in the longitudinal axis direction. With this arrangement, the wire extending from the housing does not become bulky, and operability of the operation device can be enhanced. Thus, the operation device is rather easy to use even if the operator is alone.

(10) In the operation device of any one of (1) to (9), the treatment device may have a drive shaft configured to be rotated about the longitudinal axis, and the operation device may further include a drive source configured to impart a rotational force to the drive shaft. With this arrangement, even in a situation where the wire is likely to move due to an operation by the operator for driving the drive shaft, rotation of the drive shaft, or the like, the wire is moved by the action unit with movement of the housing, and an appropriate position can be maintained.

(11) An operation device according to the present disclosure to which the proximal portion of an elongated treatment device having a lumen through which a wire extending from an indwelling unit indwelling in a living body lumen to the outside of the body passes can be joined, the operation device including: a housing including a joining portion to which the proximal portion of the treatment device can be joined; an action unit that is disposed on the proximal side of the joining portion, and configured to be brought into contact with the wire extending from the proximal portion of the treatment device, to apply, to the wire, a moving force for the wire to move in the longitudinal axis direction; an action unit drive source configured to drive the action unit; and an operation switch that is disposed so as to be operable from outside the housing, and configured to start and stop the action unit drive source.

The operation device of (11) can move the wire in the direction opposite to the movement of the housing, in accordance of an operation of the operation switch. Thus, it is possible to help prevent the wire from moving with movement of the housing and the indwelling unit from unintentionally moving with respect to the living body lumen. Thus, when the treatment device is used, the operation device can maintain an appropriate position and shape of the indwelling unit joined to the wire extending through the lumen of the treatment device.

(12) A method of operating a medical care system according to the present disclosure that includes: an indwelling device having an indwelling unit indwelling in a living body lumen, and a wire extending from the indwelling unit; an elongated treatment device having a lumen through which the wire passes; and an operation device that has the proximal portion of the treatment device joined to the operation device and configured to operate the treatment device. The method includes: inserting the treatment device into the living body lumen while the wire extending from the indwelling unit indwelling in the living body lumen to the outside of the body is accommodated in the lumen; securing the wire extending from the operation device to a surgical table or a securing target object that is an object placed on the surgical table; maintaining the securing of the wire to the surgical table or the securing target object when the operation device is moved in the distal direction; and, when the operation device is moved in the proximal direction, the wire is moved in the distal direction with respect to the operation device.

By the method of operating the medical care system of (12), the wire can be secured to the securing target object, so that unintended movement of the indwelling unit due to a force received by the indwelling unit from the blood flow or the like or a sliding force received by the wire from the treatment device or the like can be prevented. Accordingly, when the treatment device is used, the method of operating the medical care system can maintain an appropriate position and shape of the indwelling unit joined to the wire extending through the lumen of the treatment device. When the operation device is moved in the distal direction, the wire is secured to the securing target object, so that it is possible to effectively prevent the wire from moving in the distal direction together with the treatment device. Further, when the operation device is moved in the proximal direction, it can be difficult to prevent the wire from moving in the proximal direction together with the treatment device, by securing the wire to the securing target object. Therefore, the wire is moved in the distal direction relative to the operation device, so that unintended movement of the indwelling unit can be prevented.

### BRIEF DESCRIPTION OF THE DRAWINGS

FIG. 1 is a plan view of a medical care system including an operation device according to a first embodiment.

FIG. 2 is a plan view of the operation device according to the first embodiment.

FIG. 3 is a perspective view of part of the operation device according to the first embodiment.

FIG. 4 is a perspective view illustrating two rotating rollers, two meshing gears, and a wire.

FIG. 5 is an enlarged cross-sectional view of part of the housing of the operation device and a wire holding unit.

FIGS. 6A and 6B are perspective views showing part of the operation device in a transparent manner, and wherein FIG. 6A illustrates a state in which the operation device is moving in the distal direction, and FIG. 6B illustrates a state in which the operation device is moving in the proximal direction.

FIG. 7 is a plan view of an operation device according to a second embodiment.

FIG. 8 is a perspective view of part of the operation device according to the second embodiment.

FIGS. 9A and 9B are perspective views showing part of the operation device according to the second embodiment, and wherein FIG. 9A illustrates a state in which the operation device is moving in the distal direction, and FIG. 9B illustrates a state in which the operation device is moving in the proximal direction.

FIGS. 10A to 10C are plan views illustrating an operation device according to a third embodiment, and wherein FIG. 10A illustrates a state in which the position of the operation device is maintained, FIG. 10B illustrates a state in which the operation device is moving in the distal direction, and FIG. 10C illustrates a state in which the operation device is moving in the proximal direction.

FIGS. 11A and 11B are cross-sectional views illustrating modifications of the wire holding unit, and wherein FIG. 11A illustrates a first modification, and FIG. 11B illustrates a second modification.

FIG. 12 is a plan view of an operation device according to a third modification.

FIG. 13 is a perspective view illustrating a state in which a securing tool and a holding tool of a wire holding unit of a third modification are separated.

FIG. 14 is a perspective view illustrating a state in which the securing tool and the holding tool of the wire holding unit of the third modification are combined.

FIG. 15 is a cross-sectional view taken along line XV-XV defined in FIG. 14.

FIGS. 16A and 16B are cross-sectional views of the securing tool of the wire holding unit of the third modification, and wherein FIG. 16A illustrates a state in which the wire is inserted through the securing tool, and FIG. 16B illustrates a state in which the securing tool to which the wire is secured is combined with the holding tool.

FIG. 17 is a perspective view of part of a fourth modification of the operation device according to the first embodiment.

FIG. 18 is a perspective view of part of a fifth modification of the operation device according to the first embodiment.

FIG. 19 is a perspective view of part of a sixth modification of the operation device according to the first embodiment.

### DETAILED DESCRIPTION

Set forth below with reference to the accompanying drawings is a detailed description of embodiments of an operation device for operating a treatment device inserted into a living body lumen, and a method of operating a medical care system. Note that dimensional ratios in the drawings may be exaggerated and different from actual ratios, for convenience of explanation.

### First Embodiment

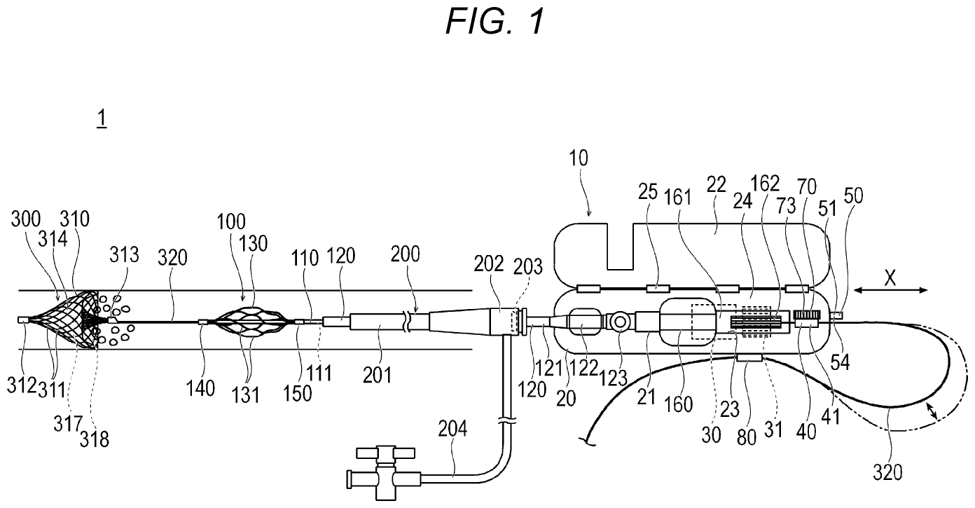

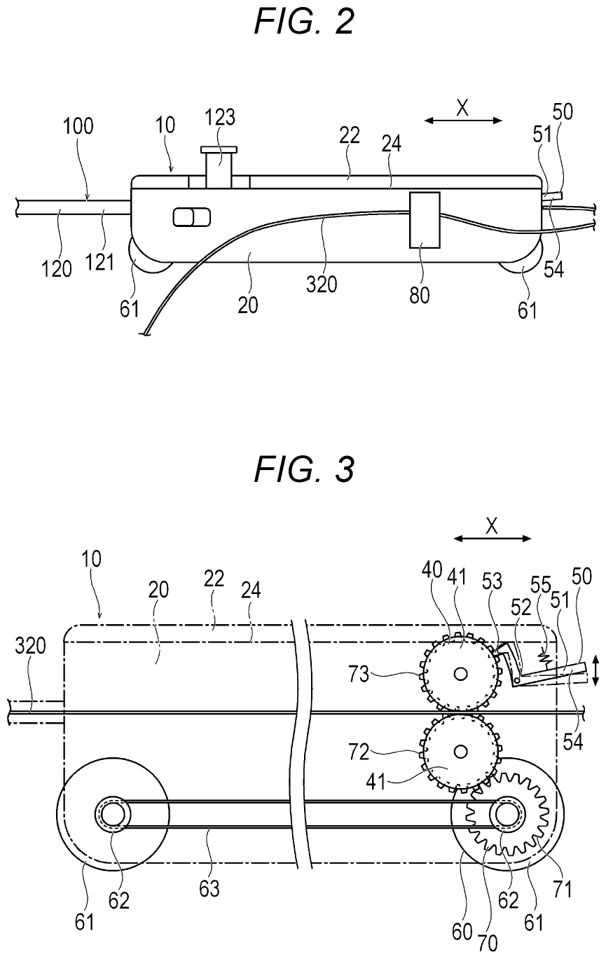

As illustrated in FIGS. 1 and 2, an operation device 10 according to a first embodiment is connected to a proximal portion of an elongated treatment device 100 inserted into a living body lumen such as a blood vessel to perform treatment, and is used to operate the treatment device 100. The treatment device 100, an introducer sheath 200, an indwelling device 300 including an indwelling unit 310 that can indwell in the living body lumen, and the operation device 10 constitute a medical care system 1. In the present specification, of the treatment device 100, the introducer sheath 200, and the indwelling device 300, the side to be inserted into the living body lumen is referred to as the “distal side”, and the operating side is referred to as the “proximal side”. Further, the direction in which the treatment device 100, the introducer sheath 200, and the indwelling device 300 extend in a longitudinal length is referred to as the longitudinal axis direction X.

Note that the living body lumen into which the medical care system 1 is inserted is not necessarily a blood vessel, but may be a vascular channel, a ureter, a bile duct, a fallopian tube, a hepatic duct, or the like, for example.

First, the treatment device 100 is described. The treatment device 100 in the present embodiment is a device that is inserted into a blood vessel to physically crush a thrombus. Crushing means breaking an object with a structure, and the shape of the structure, the breaking method, the degree of breaking, the range of breaking, the shape of the broken object, and the like are not limited to any particular shapes, breaking methods, degrees of breaking, ranges of breaking, and shapes of the broken object. The treatment device 100 includes a drive shaft 110, a suction unit 120 that sucks or suctions a crushed object, a treatment unit 130, a securing portion 140, a sliding portion 150, and a hub 160.

The drive shaft 110 is an elongated tubular member that transmits rotational force to the treatment unit 130. The proximal portion of the drive shaft 110 is rotatably attached to the hub 160. The drive shaft 110 has a lumen 111 into which a wire 320 of the indwelling device 300 can be inserted.

The drive shaft 110 has flexibility so as to be movable in a blood vessel. Further, the drive shaft 110 preferably has relatively high torsional rigidity so that rotational force can be transmitted from the distal portion to the proximal portion. The drive shaft 110 can be, for example, a metallic tubular member in which a spiral slit is formed, or a tubular member in which a plurality of metal wires is braided into a tubular shape, for example. The material forming the drive shaft 110 is preferably stainless steel or the like, for example.

The suction unit 120 includes a suction tube 121, and a suction hub 122 connected to the proximal portion of the suction tube 121. The suction tube 121 is a tubular member that has flexibility and rotatably accommodates the drive shaft 110, and is coaxially disposed outside the drive shaft 110. The lumen of the suction tube 121 can deform and accommodate the treatment unit 130. The suction hub 122 branches into a Y-like shape. The drive shaft 110 penetrates through one of the branches. The other branch is a suction port 123 to which a syringe or the like that applies a suction force can be connected. The suction hub 122 can be joined to a joining portion 21 of the operation device 10. The suction unit 120 is movable in the axis direction X with respect to the drive shaft 110.

The securing portion 140 can be a ring-shaped member that secures the treatment unit 130 to the drive shaft 110. The securing portion 140 is secured to the outer peripheral surface of the distal end of the drive shaft 110. Further, the securing portion 140 is secured to the distal end of the treatment unit 130.

The sliding portion 150 can be a ring-shaped member that is provided on the proximal side of the securing portion 140 and is disposed on the outer peripheral surface of the drive shaft 110 so as to be slidable in the axis direction X. The sliding portion 150 is secured to the proximal end of the treatment unit 130.

The treatment unit 130 is a member that expands in a blood vessel and crushes an object such as a thrombus by rotating. The treatment unit 130 is secured to the distal portion of the drive shaft 110. The treatment unit 130 can include a plurality of deformable wire rods 131 arranged in a circumferential direction. Each wire rod 131 is preferably formed with a material having shape memory properties so that the wire rod 131 can be elastically deformed to a relatively large extent. The distal end of each wire rod 131 is secured to the securing portion 140. The proximal end of each wire rod 131 is secured to the sliding portion 150. The substantially central portions, which are curved, of the respective wire rods 131 are arranged in the circumferential direction at positions distant in the radial direction outwardly from the drive shaft 110. With this configuration, the treatment unit 130 uniformly expands in the circumferential direction as a whole. The treatment unit 130 expands in the radial direction in the natural state where no external force acts on the treatment unit 130. When the drive shaft 110 rotates, the treatment unit 130 also rotates accordingly, and can crush a thrombus in the blood vessel or stir crushed thrombi. The treatment unit 130 contracts in the radial direction, receiving a force for separating both ends in the longitudinal axis direction X. Alternatively, the treatment unit 130 contracts in the radial direction, receiving a force applied from the outside to the inside in the radial direction.

Note that the form of the treatment unit 130 is not limited to this. For example, instead of being formed with the plurality of wire rods 131, the treatment unit 130 may include abrasive grains or a blade, to have a crushing function. Alternatively, the treatment unit 130 may not be expandable and contractible.

The hub 160 is a member that can be gripped and operated by the operator, and can be joined to the operation device 10. The hub 160 includes a hub main unit 161 in which the proximal portion of the drive shaft 110 is disposed, and a driven gear 162 that is rotatably supported inside the hub main unit 161 and is secured to the drive shaft 110.

The hub main unit 161 rotatably accommodates the proximal portion of the drive shaft 110 and the driven gear 162. Part of the driven gear 162 is placed inside the hub main unit 161, and part of the driven gear 162 is exposed to the outside through the hub main unit 161.

The introducer sheath 200 includes an elongated sheath 201, an introducer hub 202 joined to the proximal portion of the sheath 201, a valve member 203 disposed in the introducer hub 202, and a port 204 branching from the introducer hub 202. The introducer sheath 200 is inserted percutaneously into a blood vessel, to form an access path to the blood vessel.

The sheath 201 is a tubular member that is inserted into a blood vessel, to form an access path to the blood vessel. Alternatively, the sheath 201 may be capable of contracting and accommodating the indwelling unit 310 of the indwelling device 300. The sheath 201 has flexibility so as to be movable in a blood vessel.

Next, the indwelling device 300 is described. The indwelling device 300 is a device that has the indwelling unit 310 indwelling in a living body lumen, and in the present embodiment, is a device that has a filter as the indwelling unit 310 indwelling in a blood vessel. The indwelling device 300 includes the indwelling unit 310, and the wire 320 extending from the indwelling unit 310 to the outside of the body. The wire 320 enables collection of the indwelling unit 310 indwelling in the living body lumen, and also has a function as a guide wire that guides another device (for example, the treatment device 100) to the target position.

The indwelling unit 310 can be a filter that gathers objects such as thrombi crushed by the treatment device 100 and flowing together with blood. The indwelling unit 310 includes a plurality of flexibly deformable linear members 311 braided in a net to form a tubular member, a distal-side joining portion 312, and a proximal-side joining portion 313 joined to the wire 320. An internal space 317 is formed inside the indwelling unit 310 forming a tubular member. The plurality of linear members 311 is braided to have openings 314 between the linear members 311. Note that the indwelling unit 310 is not necessarily a filter as long as it can indwell in a living body lumen, and may be, for example, a balloon or a stent-like member.

The distal-side joining portion 312 secures the distal ends of the plurality of linear members 311. The proximal-side joining portion 313 secures the proximal ends of the plurality of linear members 311 and the distal end of the wire 320.

In the natural state in which no external force acts, the indwelling unit 310 is folded back in the axis direction by the elastic force (restoring force) of the linear members 311. When the indwelling unit 310 is folded back, the proximal-side joining portion 313 and the distal-side joining portion 312 approach each other. In the folded state, the portions of the plurality of linear members 311 joined to the proximal-side joining portions 313 are located in the portions of the plurality of linear members 311 joined to the distal-side joining portion 312. The portions of the linear members 311 joined to the proximal-side joining portion 313 are arranged in a concave shape that is open to the proximal side, and forms a collection space 318 for collecting thrombi and the like.

The number of the linear members 311 is not limited to any particular number. The condition for braiding the linear members 311 is not limited to any particular condition. The material forming the linear members 311 is preferably a flexible material. Examples of preferred materials forming the linear members 311 can include a shape memory alloy to which a shape memory effect and superelasticity are imparted by heat treatment, stainless steel, tantalum (Ta), titanium (Ti), white silver (Pt), gold (Au), tungsten (W), polyolefins such as polyethylene and polypropylene, polyamides, polyesters such as polyethylene terephthalate, fluorine-based polymers such as ETFE (tetrafluoroethylene-ethylene copolymer), PEEK (polyetheretherketone), and polyimides.

The material forming the distal-side joining portion 312 and the proximal-side joining portion 313 are not limited to any particular material. For example, stainless steel, polyetheretherketone (PEEK), or the like can be suitably used for the material forming the distal-side joining portion 312.

When accommodated in the sheath 201, the indwelling unit 310 is elastically deformed to be in a contracted state having a relatively small outer diameter. When the indwelling unit 310 is in a contracted state, the proximal-side joining portion 313 and the distal-side joining portion 312 are separated from each other.

When released from the sheath 201, the indwelling unit 310 indwells in a blood vessel in a shape close to the natural state, as illustrated in FIG. 1. In practice, the indwelling unit 310 indwells on the blood vessel wall, while contracting to some extent in the radial direction from the natural state so as to generate a pressing force onto the blood vessel wall by its own expansion force.

Note that the indwelling unit 310 may not be in a folded state immediately after being released from the sheath 201 in the blood vessel. In this case, after the indwelling unit 310 indwells in the blood vessel, the indwelling unit 310 can be pushed toward the distal side by the sheath 201 or the like, and be folded back.

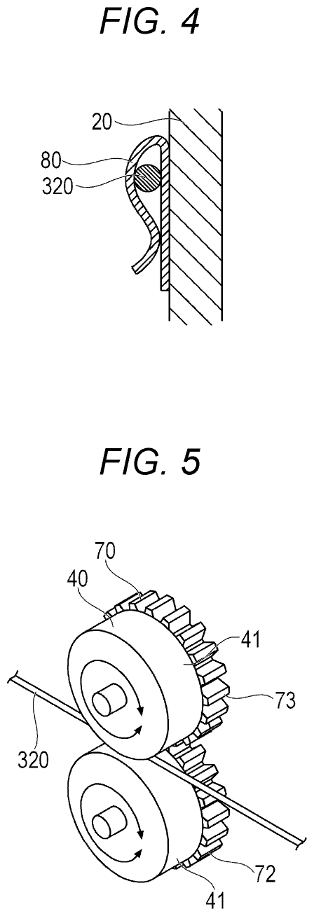

Next, the operation device 10 is described. As illustrated in FIGS. 1 to 5, the operation device 10 is a unit that has the proximal portion of the treatment device 100 joined to the operation device 10, and operates the treatment device 100. In the present embodiment, the operation device 10 is a drive unit that includes a drive source 30 and drives the treatment device 100 having a crushing function. The operation device 10 is a portable device that can be lifted up and operated by an operator.

The operation device 10 can include: a housing 20 including the joining portion 21 capable of joining the hub 160 and the suction hub 122 of the treatment device 100; a lid portion 22 that can cover the joining portion 21 of the housing 20; the drive source 30 such as a motor for driving the drive shaft 110; and a drive gear 31 that is rotated by the drive source 30. The operation device 10 can further include: an action unit 40 that applies a moving force to the wire 320; a limiting mechanism 50 capable of limiting operations of the action unit 40; a movement detection unit 60 that detects movement of the housing 20; a transmission unit 70 that operates the action unit 40 in accordance with a result of the detection performed by the movement detection unit 60; and a wire holding unit 80 disposed outside the housing 20.

The housing 20 has a joining surface 24 in which a passage 23 is formed, and the joining portion 21 is formed in part of the passage 23. The treatment device 100 and the indwelling device 300 extend through the passage 23. The lid portion 22 can be joined to the housing 20 by a hinge 25 so that the lid portion 22 can be opened and closed. The lid portion 22 can cover the joining surface 24 of the housing 20, or can expose the joining surface 24. The drive source 30 and the drive gear 31 are disposed in the housing 20.

As illustrated in FIGS. 1, 3, and 5, the action unit 40 is disposed on the proximal side of the joining portion 21, and can be brought into contact with the wire 320 extending in the proximal direction from the proximal end of the drive shaft 110, to apply a moving force in the longitudinal axis direction X to the wire 320. The action unit 40 can include two rotating rollers 41 having an outer peripheral surface that can come into contact with the wire 320. The two rotating rollers 41 are rotatable about a rotation axis perpendicular to the extending direction of the passage 23. The rotation axes of the two rotating rollers 41 are parallel to each other, and the passage 23 is interposed between the rotation axes. The two rotating rollers 41 are disposed symmetrically with respect to the passage 23, with a gap capable of pinching the wire 320 extending through the passage 23 without sliding. At least part of at least the outer peripheral surfaces of the rotating rollers 41 is preferably formed with a rubber material, a hard material having a plurality of irregularities, or the like so as to be less slippery. The rubber material is natural rubber such as latex rubber, or synthetic rubber such as silicone rubber, urethane rubber, or butadiene rubber, for example. On the outer peripheral surfaces of the two rotating rollers 41, a groove for stably holding the wire 320 may be formed to extend in the circumferential direction. The layout of the two rotating rollers 41 is not particularly limited as long as a force can be applied to the wire 320. For example, when the joining surface 24 is viewed from above, the two rotating rollers 41 may be interposed between the passage 23 vertically with respect to the passage 23, or may be interposed between the passage 23 horizontally with respect to the passage 23. Between the outer peripheral surfaces of the two rotating rollers 41, the wire 320 can be disposed to be inserted from one end side in the axis direction of the two rotating rollers 41.

The action unit 40 may be disposed in the housing 20 or may be disposed outside the housing 20 (for example, the outside on the proximal side of the housing 20).

The movement detection unit 60 detects a movement amount (displacement) and a movement direction (the distal direction or the proximal direction) of the housing 20. The movement detection unit 60 can include: two detecting rollers (or detection rollers) 61 whose outer peripheral surfaces partially protrude from the housing 20 at positions on the distal side and the proximal side of the housing 20; two pulleys 62 that rotate integrally with the respective detecting rollers 61; and a belt 63 stretched around the two pulleys 62.

As for one of the detecting rollers 61, part of the outer peripheral surface protrudes from the housing 20 toward the distal side and the lower side at a position on the distal side and the lower side of the housing 20 (the side opposite to the upper side on which the joining surface 24 is provided). As for the other one of the detecting rollers 61, part of the outer peripheral surface protrudes from the housing 20 toward the distal side and the lower side at a position on the proximal side and the lower side of the housing 20. With this arrangement, even when a contact target object 500 has various undulations, one of the two detecting rollers 61 can be brought into contact with the contact target object 500. The outer peripheral surfaces of the two detecting rollers 61 come into contact with the contact target object 500, which is the patient, the surgical table, the drape, or an object placed on the surgical table, so that the two detecting rollers 61 rotate by receiving a rotational force from the contact target object 500 when the housing 20 moves. The two detecting rollers 61 are rotatable about rotation axes that are perpendicular to the extending direction of the passage 23, and are parallel to the rotation axes of the rotating rollers 41. Note that the rotation axes of the detecting rollers 61 may not be parallel to the rotation axes of the rotating rollers 41. At least the outer peripheral surfaces of the detecting rollers 61 are preferably formed with a rubber material, a hard material having a plurality of irregularities, or the like so as to be less slippery. The rubber material can be, for example, a natural rubber such as latex rubber, or synthetic rubber such as silicone rubber, urethane rubber, or butadiene rubber.

The two pulleys 62 are secured so as to rotate coaxially with the two respective detecting rollers 61. Since the two pulleys 62 are connected by the belt 63, the pulleys 62 rotate in the same direction.

The movement amounts to be detected by the two respective detecting rollers 61 connected by the belt 63 needs to match. Therefore, the circumferential length of each detecting roller 61 and the circumferential length of each pulley 62 are appropriately set so that the movement amounts to be detected by the two detecting rollers 61 match. For example, if setting is performed so that the circumferential lengths of the two pulleys 62 match and the circumferential lengths of the two detecting rollers 61 match, the two detecting rollers 61 can detect the same movement amounts from movement of the housing 20. As the two pulleys 62 are connected by the belt 63, if at least one of the two detecting rollers 61 can come into contact with the contact target object 500, the movement detection unit 60 can detect a movement amount and a movement direction of the housing 20.

The number of detecting rollers 61 may be one, or may be three or more. Further, a flexible annular member in a caterpillar-like form may be disposed so as to surround the entire plurality of detecting rollers 61. In this case, if any portion of the caterpillar-like flexible annular member can come into contact with the contact target object 500, the movement detection unit 60 can detect a movement amount and a movement direction of the housing 20. Further, the form of the movement detection unit 60 is not limited to any particular one, as long as a movement amount and a movement direction of the housing 20 can be detected. For example, an acceleration sensor or the like may be included.

The transmission unit 70 is a unit that operates the action unit 40 in accordance with a result of detection performed by the movement detection unit 60. The transmission unit 70 can include: a first gear 71 that rotates integrally and coaxially with one of the two detecting rollers 61 (the detecting roller 61 on the proximal side in the present embodiment); a second gear 72 that rotates integrally and coaxially with one of the rotating rollers 41; and a third gear 73 that rotates integrally and coaxially with the other one of the rotating rollers 41. The first gear 71 and the second gear 72 mesh with each other, and the second gear 72 and the third gear 73 mesh with each other. The second gear 72 and the third gear 73 are formed with the same number of teeth, to rotate the two rotating rollers 41 having symmetrical shapes in opposite directions at the same angular velocity. In a case where the circumferential lengths of the two rotating rollers 41 are different, the second gear 72 and the third gear 73 may be formed with different numbers of teeth. The configuration of the transmission unit 70 is not limited to any particular one, as long as rotation detected by the movement detection unit 60 can be transmitted to the action unit 40. Therefore, the transmission unit 70 may be formed only with gears without including the belt 63 and the pulley 62, or may be formed with a belt and pulleys without including any gear.

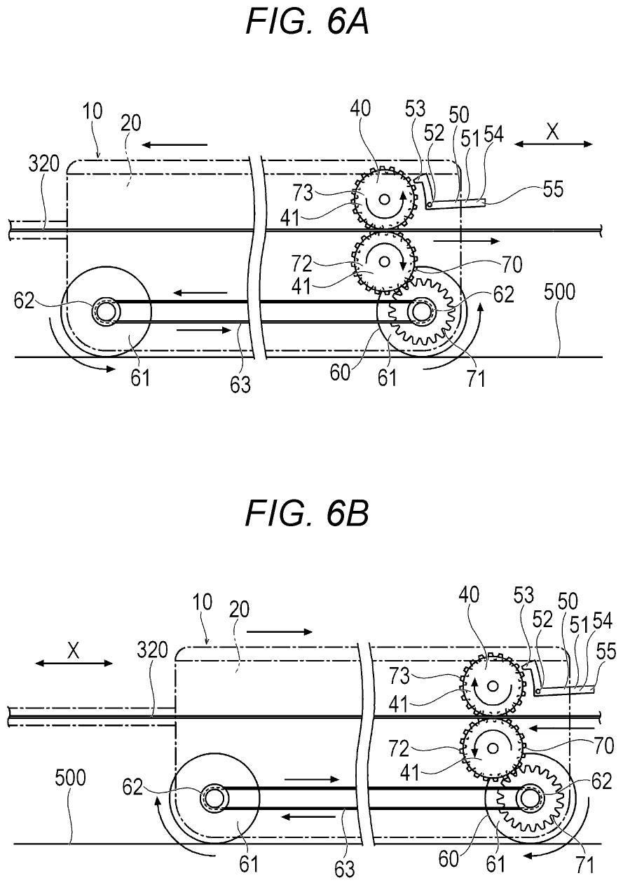

When the housing 20 moves to the distal side while at least one of the detecting rollers 61 is in contact with the contact target, as illustrated in FIG. 6A, the detecting rollers 61 and the first gear 71 rotate counterclockwise on the paper surface of this drawing, the second gear 72 meshing with the first gear 71 and the lower rotating roller 41 integrated with the second gear 72 rotate clockwise, and the third gear 73 meshing with the second gear 72 and the upper rotating roller 41 integrated with the third gear 73 rotate counterclockwise. The wire 320 interposed between the lower rotating roller 41 rotating clockwise and the upper rotating roller 41 rotating counterclockwise receives a moving force from the two rotating rollers 41 so as to move in the proximal direction with respect to the housing 20. The movement amounts detected by the detecting rollers 61 match the displacement amounts given to the wire 320 by the two rotating rollers 41. Further, the moving direction detected by the detecting rollers 61 and the direction of displacement given to the wire 320 by the two rotating rollers 41 are opposite directions. Because of this, when the housing 20 moves in the distal direction by a predetermined distance, the wire 320 is moved in the proximal direction by the same distance with respect to the housing 20 by the rotating rollers 41.

Further, when the housing 20 moves to the proximal side while at least one of the detecting rollers 61 is in contact with the contact target, as illustrated in FIG. 6B, the detecting rollers 61 and the first gear 71 rotate clockwise on the paper surface of this drawing, the second gear 72 meshing with the first gear 71 and the lower rotating roller 41 integrated with the second gear 72 rotate counterclockwise, and the third gear 73 meshing with the second gear 72 and the upper rotating roller 41 integrated with the third gear 73 rotate clockwise. The wire 320 interposed between the lower rotating roller 41 rotating counterclockwise and the upper rotating roller 41 rotating clockwise receives a moving force from the two rotating rollers 41 so as to move in the distal direction with respect to the housing 20. The movement amounts detected by the detecting rollers 61 match the displacement amounts given to the wire 320 by the two rotating rollers 41. Further, the moving direction detected by the detecting rollers 61 and the direction of displacement given to the wire 320 by the two rotating rollers 41 are opposite directions. Because of this, when the housing 20 moves in the proximal direction by a predetermined distance, the wire 320 is moved in the distal direction by the same distance with respect to the housing 20 by the rotating rollers 41.

Therefore, even when the housing 20 moves to the distal side or the proximal side, the wire 320 does not move together with the housing 20, and the absolute position in the longitudinal axis direction X hardly changes. Thus, the indwelling unit 310 secured to the distal portion of the wire 320 can be kept from moving with respect to the blood vessel.

The limiting mechanism 50 is a mechanism that limits rotation of the rotating rollers 41. As illustrated in FIG. 3, the limiting mechanism 50 can include: a limiting member 51 that can be rotated with respect to the housing 20 by a fulcrum 52; and a biasing member 55 that biases the limiting member 51. On one end side of the limiting member 51, a protruding portion 53 that can enter the tooth grooves of the third gear 73 (or the second gear 72) and retract from the tooth grooves is formed. On the opposite side of the fulcrum 52 from the protruding portion 53, a limiting operation portion 54 that protrudes outwardly from the housing 20 and can be operated by the operator is formed. The biasing member 55 can be a spring member, for example, and has one end secured to the housing 20, and the other end secured to the limiting member 51. The biasing member 55 biases the limiting member 51 so that the protruding portion 53 of the limiting member 51 moves toward the tooth grooves of the third gear 73. With the limiting member 51 being biased by the biasing member 55, the protruding portion 53 enters the tooth groove of the third gear 73. With this arrangement, the rotating rollers 41 become unable to rotate, and the wire 320 in contact with the rotating rollers 41 becomes unable to move with respect to the housing 20. When the operator presses the limiting operation portion 54, the limiting member 51 rotates about the fulcrum 52 against the biasing force of the biasing member 55, and the protruding portion 53 retracts from the tooth grooves of the rotating rollers 41. With this arrangement, the rotating rollers 41 become able to rotate, and the wire 320 in contact with the rotating rollers 41 becomes able to move respect to the housing 20. When the operator takes his/her hand off the limiting operation portion 54, the limiting member 51 rotates by the biasing force received from the biasing member 55, and the protruding portion 53 enters the tooth grooves of the third gear 73. With this arrangement, the rotating rollers 41 become unable to rotate, and the wire 320 in contact with the rotating rollers 41 becomes unable to move respect to the housing 20.

Since the limiting mechanism 50 can limit movement of the wire 320, it is possible to prevent thrombi from accumulating in the indwelling unit 310 serving as a filter, and the wire 320 from moving in the distal direction by the blood flow in the distal direction without permission. In a case where the operator wishes to move the operation device 10 without moving the indwelling device 300, the operator presses the limiting operation portion 54 to cancel the limiting mechanism 50. Thus, the operator can operate the operation device 10 without moving the indwelling device 300.

As illustrated in FIGS. 1, 2, and 5, the wire holding unit 80 is an elastically deformable clip-shaped member disposed on the outer surface of the housing 20. The wire 320 extending in the proximal direction from the proximal end of the housing 20 is interposed and held while being bent in the distal direction. It is preferable that the wire holding unit 80 can limit movement of the wire 320 in the longitudinal axis direction X while rotatably holding the wire 320. With this arrangement, when the operation device 10 moves to the distal side, the wire 320 extends from the operation device 10 to the proximal side, so that the length of the wire 320 between the proximal end of the housing 20 and the wire holding unit 80 increases, and the arc of the wire 320 becomes larger (see the two-dot-and-dash line in FIG. 1).

Next, a method of use of the medical care system 1 including the operation device 10 according to the first embodiment is described.

The operator inserts the introducer sheath 200 into a blood vessel percutaneously on the upstream side (proximal side) of the lesion in the blood vessel, and inserts the indwelling device 300 accommodated in the suction tube 121 of the suction unit 120 by contracting the indwelling unit 310, percutaneously into the vein via the introducer sheath 200. Next, the operator releases the indwelling unit 310 from the suction tube 121 at a position beyond the lesion such as a thrombus (a position on the downstream side of the lesion). When released from the suction tube 121, the indwelling unit 310 expands so as to generate a pressing force against the blood vessel wall by its own expanding force, and indwells on the blood vessel wall. The indwelling unit 310 is preferably in a folded state.

Next, as illustrated in FIG. 1, the operator inserts the treatment device 100 accommodated in the suction tube 121 of the suction unit 120 by contracting the treatment unit 130, into the blood vessel along the wire 320 extending from the indwelling unit 310 to the outside of the body, to reach the vicinity of the lesion. The wire 320 slides in the lumen 111 of the treatment device 100. Next, the operator moves the suction unit 120 to the proximal side with respect to the drive shaft 110, and releases the treatment unit 130 secured to the distal portion of the drive shaft 110 from the suction tube 121 of the suction unit 120. When released from the suction tube 121, the treatment unit 130 returns to its original shape by its own elastic force.

Next, the operator joins the hub 160 and the suction hub 122 of the treatment device 100 to the joining portion 21 of the operation device 10. Further, the wire 320 extending from the proximal end of the hub 160 of the treatment device 100 is placed between the two rotating rollers 41. When the hub 160 of the treatment device 100 is joined to the joining portion 21, the drive gear 31 of the operation device 10 meshes with the driven gear 162 of the treatment device 100. Thus, the drive shaft 110 can be rotated by rotation of the drive source 30. The drive source 30 can reciprocate in the rotation direction. Note that the drive source 30 is not limited to a reciprocating one, and the drive source 30 may rotate in one direction.

The operator also causes the wire holding unit 80 to hold the wire 320 extending in the proximal direction from the proximal end of the housing 20 while being bent in the distal direction. The wire holding unit 80 limits movement of the wire 320 in the longitudinal axis direction X while rotatably holding the wire 320. With this arrangement, when the entire operation device 10 moves to the distal side, the wire 320 extends from the operation device 10 to the proximal side, so that the length of the wire 320 between the proximal end of the housing 20 and the wire holding unit 80 increases, and the arc of the wire 320 becomes larger. Because of this, the operation device 10 is relatively easy to use even in a case where the wire 320 is several meters long on the proximal side of the housing 20. Accordingly, the operator can operate the wire 320 without requiring an assistant doctor or the like to hold the wire 320. Thus, the wire holding unit 80 can enhance operability of the elongated wire 320 on the proximal side of the housing 20, and can avoid contamination of the wire 320 due to contact with a dirty region.

As the limiting mechanism 50 that limits unintended rotation of the rotating rollers 41 is provided in the operation device 10, movement of the wire 320 interposed between the rotating rollers 41 in the longitudinal axis direction X is limited. Accordingly, it is possible to prevent the indwelling unit 310 and the wire 320 from unintentionally moving in the distal direction and the proximal direction due to the blood flow in the distal direction acting on the indwelling unit 310, movement of each device (the treatment device 100, the suction unit 120, the operation device 10, the indwelling device 300, and the introducer sheath 200), or the sliding between each device and the wire 320.

In a case where the operator wishes to move the operation device 10 without moving the indwelling device 300, the operator presses the limiting operation portion 54 to cancel the limiting mechanism 50, as illustrated in FIG. 3. Thus, the operation device 10 can be put into an operable state without any movement of the indwelling device 300.

Next, as illustrated in FIGS. 6A and 6B, the operator brings the outer peripheral surfaces of the detecting rollers 61 of the operation device 10 into contact with the patient, the surgical table, the drape, or the contact target object 500 that is an object placed on the surgical table. For example, when the treatment device 100 is inserted in a direction from the skin of the thigh of the patient in a recumbent position toward the heart through the femoral vein, the operator can bring the detecting roller 61 into contact with the lower limbs or a drape covering the lower limbs of the patient. Since the operator only needs to select at least one of the detecting rollers 61 on the distal side and the proximal side of the housing 20 in accordance with the undulations of the contact target object 500, movement of the housing 20 can be reliably and accurately detected.

Next, the operator actuates the drive source 30, to rotate the drive shaft 110 and the treatment unit 130. The operator then moves the operation device 10 alternately in the distal direction and the proximal direction, to move the treatment unit 130 in the blood vessel alternately in the distal direction and the proximal direction, and crush the thrombus. When moving the operation device 10 in the longitudinal axis direction X (the distal direction and the proximal direction), the operator brings the outer peripheral surfaces of the detecting rollers 61 into contact with the contact target object 500. In the present embodiment, in a case where the operation device 10 is moved in the longitudinal axis direction X while the detecting rollers 61 of the movement detection unit 60 are in contact with the contact target object 500, as described above, the rotating rollers 41 of the action unit 40 rotate in accordance with the movement amount and the movement direction detected by the movement detection unit 60. The absolute position of the wire 320 moving by receiving a force from the rotating rollers 41 hardly changes in the longitudinal axis direction X. Therefore, even if the treatment device 100 and the operation device 10 move, and a sliding force acts on the wire 320, the indwelling unit 310 secured to the distal portion of the wire 320 is less likely to be displaced, and the shape developed to cover the blood vessel lumen can be prevented from being canceled (or changed). Furthermore, since the limiting mechanism 50 that limits unintended rotation of the rotating rollers 41 is provided in the operation device 10, it is possible to prevent thrombi from accumulating in the indwelling unit 310 as a filter, and the wire 320 from unintentionally moving in the distal direction due to the blood flow in the distal direction. In a case where the operator wishes to move the operation device 10 without moving the indwelling device 300, the operator presses the limiting operation portion 54 to cancel the limiting mechanism 50. Thus, the operator can operate the operation device 10 without moving the indwelling device 300.

After completing the crushing of the thrombi by the treatment device 100, the operator stops the drive source 30 to stop the rotation of the drive shaft 110 and stop the movement of the housing 20. Subsequently, the operator removes the treatment device 100 from the operation device 10. Further, the operator removes the wire 320 from the action unit 40. The drive shaft 110 is moved relative to the suction tube 121 in the proximal direction, and the treatment unit 130 is accommodated and contracted in the suction tube 121. Further, the operator moves the drive shaft 110 in the proximal direction with respect to the suction tube 121, and removes the treatment device 100 from the suction tube 121 while leaving the suction tube 121 in the blood vessel.

Next, the operator cancels the pressing of the limiting operation portion 54. As the pressing of the limiting operation portion 54 is canceled, the protruding portion 53 of the limiting mechanism 50 enters the tooth grooves of the third gear 73, and unintended rotation of the rotating rollers 41 is limited. Thus, unintended movement of the wire 320 and the indwelling unit 310 can be prevented. Further, it is possible to appropriately maintain the shape of the indwelling unit 310 that is collecting thrombi, and prevent the thrombi from flowing beyond the indwelling unit 310.

Next, the operator places the distal end of the suction tube 121 of the suction unit 120 near the position at which thrombi are being collected in the indwelling unit 310. At this point of time, a syringe or the like is connected to the suction port 123 of the suction unit 120, to apply a suction force. In this manner, the thrombi collected by the indwelling unit 310 can be sucked (or suctioned) and removed from the opening at the distal end of the suction tube 121. As a result, the proximal portion of the indwelling unit 310 forming the collection space 318 alternately moves in the distal direction and the proximal direction, and the thrombi collected by the indwelling unit 310 float from the indwelling unit 310, and are easily sucked (or suctioned) from the distal end of the suction tube 121.

After completing the suction of the thrombi with the suction unit 120, the operator pushes the suction unit 120 in the distal direction while pulling the wire 320 in the proximal direction, and accommodates the indwelling unit 310 in the suction tube 121 while contracting the indwelling unit 310. After that, the operator removes the suction unit 120, the introducer sheath 200, and the indwelling device 300 from the blood vessel.

As movement of the wire 320 and the indwelling unit 310 can be limited by the operation device 10 during the above-described procedures, the operator can take his/her hand off the wire 320, as necessary.

As described above, the operation device 10 according to the first embodiment is the operation device 10 to which the proximal portion of the elongated treatment device 100 having the lumen 111 through which the wire 320 extending from the indwelling unit 310 indwelling in a living body lumen to the outside of the body passes can be joined, and includes: the housing 20 including the joining portion 21 to which the proximal portion of the treatment device 100 can be joined; and the action unit 40 that is disposed on the proximal side of the joining portion 21, and is brought into contact with the wire 320 extending from the proximal portion of the treatment device 100, to apply, to the wire 320, the moving force for the wire 320 to move in the longitudinal axis direction X. With this arrangement, the operation device 10 can move the wire 320 in the longitudinal axis direction X with the action unit 40, and thus, it is possible to prevent the indwelling unit 310 from unintentionally moving with respect to the living body lumen due to movement of the wire 320, a force received from the blood flow, or the like, and the wire 320 from unintentionally loosening. Thus, when the treatment device 100 is used, the operation device 10 can maintain appropriate positions and/or shapes of the indwelling unit 310 joined to the wire 320 extending through the lumen 111 of the treatment device 100, and the wire 320.

The action unit 40 includes at least one rotating roller 41 having an outer peripheral surface that can come into contact with the wire 320. With this arrangement, the operation device 10 can reliably and highly accurately apply a moving force to the wire 320 through the rotating rollers 41. As the rotating rollers 41 can be arranged in a space-saving manner, the operation device 10 can be prevented from becoming larger in size.

At least part of the outer peripheral surfaces of the rotating rollers 41 is formed with a rubber material. With this arrangement, the rotating rollers 41 are less likely to slip even on the wire 320 having a thin and slippery surface, and a force can be reliably applied to the wire 320.

The operation device 10 includes the limiting mechanism 50 capable of limiting rotation of the rotating rollers 41. With this arrangement, the operation device 10 can limit rotation of the rotating rollers 41 holding the wire 320, to restrict the wire 320 from unintentionally moving with respect to the housing 20. Therefore, the operation device 10 can help prevent the indwelling unit 310 in a blood vessel from unintentionally moving upon receipt of the blood flow, for example, by limiting movement of the wire 320 with the limiting mechanism 50.

The operation device 10 may include the movement detection unit 60 that detects a movement amount in the longitudinal axis direction X and a movement direction of the housing 20. With this arrangement, in the operation device 10, the movement direction and the movement amount of the wire 320 that are caused by movement of the housing 20 in the action unit 40 necessary for maintaining the appropriate positions and shapes of the indwelling unit 310 and the wire 320 can be specified by the movement detection unit 60.

The movement detection unit 60 may have at least one detecting roller 61 that can rotate with respect to the housing 20, and at least part of the outer peripheral surface of the detecting roller 61 may be located outside the housing 20. With this arrangement, when the operator wishes to move the housing 20, the detecting rollers 61 are brought into contact with the contact target object 500 such as the patient, the surgical table, or an object placed on the surgical table, to move the housing 20. Thus, the movement amount and the movement direction of the housing 20 can be reliably and highly accurately detected.

The operation device 10 includes the transmission unit 70 that actuates the action unit 40 to move the wire 320 in the direction opposite to the movement direction by a distance corresponding to the movement amount detected by the movement detection unit 60. With this arrangement, the operation device 10 can move the wire 320 in the direction opposite to the movement of the housing 20. Thus, it is possible to automatically prevent the wire 320 from moving with movement of the housing 20 and the indwelling unit 310 from unintentionally moving with respect to a living body lumen, and prevent the wire 320 from unintentionally loosening. Thus, when the treatment device 100 is used, the operation device 10 can effectively maintain appropriate positions and shapes of the indwelling unit 310 joined to the wire 320 extending through the lumen 111 of the treatment device 100, and the wire 320.

The transmission unit 70 may have at least one gear that transmits rotation detected by the detecting rollers 61 to the action unit 40. With this arrangement, the operation device 10 can reliably and highly accurately transmit rotation detected by the movement detection unit 60 to the action unit 40.

The operation device 10 may include the wire holding unit 80 that is disposed outside the housing 20 and can holding the wire 320. The wire holding unit 80 may be capable of rotating about the longitudinal axis direction X while limiting movement of the wire 320 in the longitudinal axis direction X. With this arrangement, the wire 320 extending from the housing 20 does not become bulky, and operability of the operation device 10 can be enhanced. Thus, the operation device 10 is rather easy to use even if the operator is alone.

The treatment device 100 may have the drive shaft 110 that is rotatable about the longitudinal axis, and the operation device 10 may have the drive source 30 that imparts a rotational force to the drive shaft 110. With this arrangement, even in a situation where the wire 320 is likely to move due to an operation by the operator for driving the drive shaft 110, rotation of the drive shaft 110, or the like, the wire 320 is moved by the action unit 40 with movement of the housing 20, and an appropriate position can be maintained.

### Second Embodiment

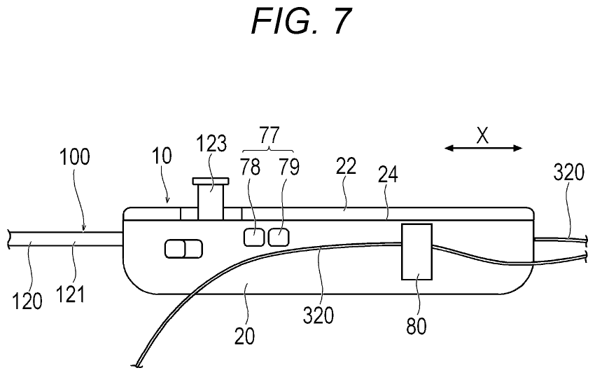

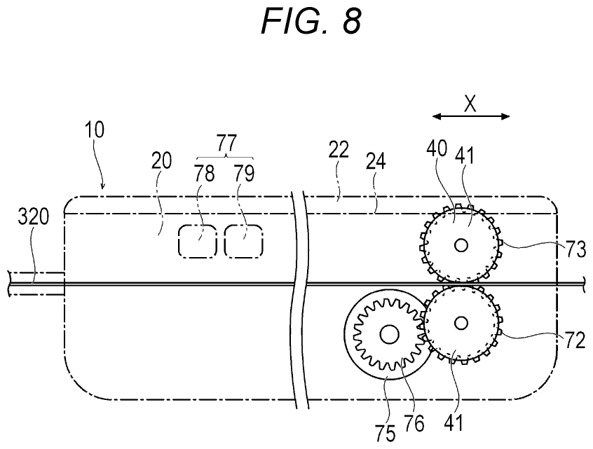

As illustrated in FIGS. 7 and 8, an operation device 10 according to a second embodiment differs from that of the first embodiment in that a drive force of the action unit 40 is obtained by an action unit drive source 75 such as a motor that can be operated by the operator as appropriate. Note that components having the same functions as those of the first embodiment are denoted by the same reference numerals as those used in the first embodiment, and explanation of them is not made herein.

The operation device 10 can include: the action unit drive source 75 that drives the rotating rollers 41 of the action unit 40; a fourth gear 76 that transmits a drive force of the action unit drive source 75 to the second gear 72 (or the third gear 73); and operation switches 77 that can be operated to start and stop the action unit drive source 75. For example, the operation switches 77 can include: a first button 78 that rotates the rotating rollers 41 so as to move the wire 320 in the distal direction with respect to the housing 20; and a second button 79 that rotates the rotating rollers 41 so as to move the wire 320 in the proximal direction with respect to the housing 20. The action unit drive source 75 secures the rotating rollers 41 so as not to rotate while any of the buttons is not pressed.

Next, a method of use of a medical care system 1 including the operation device 10 according to the second embodiment is described.

As in the first embodiment, the operator first inserts the introducer sheath 200, the indwelling device 300, and the treatment device 100 into a blood vessel, and connects the operation device 10 to the treatment device 100. Accordingly, between the two rotating rollers 41, the wire 320 extending from the proximal end of the hub 160 of the treatment device 100 is placed. Also, the drive gear 31 of the operation device 10 meshes with the driven gear 162 of the treatment device 100. Thus, the drive shaft 110 can be rotated by rotation of the drive source 30.

Next, the operator actuates the drive source 30, to rotate the drive shaft 110 and the treatment unit 130. The operator then moves the operation device 10 alternately in the distal direction and the proximal direction, to crush a thrombus with the treatment unit 130 in the blood vessel.

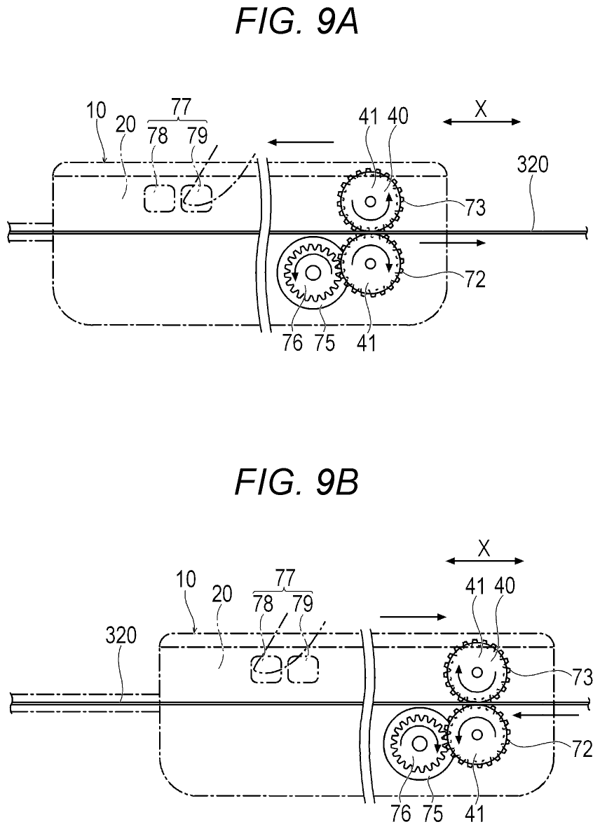

As the operation device 10 moves in the longitudinal axis direction X, the operator operates the operation switches 77. Specifically, when moving the operation device 10 in the distal direction, as illustrated in FIG. 9A, the operator presses the second button 79 of the operation switches 77, to actuate the action unit 40 and move the wire 320 in the proximal direction with respect to the housing 20. With this arrangement, the wire 320 can be moved in the proximal direction with respect to the operation device 10, in accordance with the amount of movement of the operation device 10 in the distal direction. As a result, even when the operation device 10 moves, the wire 320 is kept from moving in the distal direction together with the operation device 10 by sliding with the operation device 10. Therefore, the indwelling unit 310 secured to the distal portion of the wire 320 is less likely to be displaced, and the shape developed to cover the blood vessel lumen can be prevented from being canceled (or changed).

Also, when moving the operation device 10 in the proximal direction, as illustrated in FIG. 9B, the operator presses the first button 78 of the operation switches 77, to actuate the action unit 40 and move the wire 320 in the distal direction with respect to the housing 20. With this arrangement, the wire 320 can be moved in the distal direction with respect to the operation device 10, in accordance with the amount of movement of the operation device 10 in the proximal direction. As a result, even when the operation device 10 moves, the wire 320 is kept from moving in the proximal direction together with the operation device 10 by sliding with the operation device 10. Therefore, the indwelling unit 310 secured to the distal portion of the wire 320 is less likely to be displaced, and the shape developed to cover the blood vessel lumen can be prevented from being canceled (or changed).

Further, when maintaining the operation device 10 at a constant position, the operator does not press the operation switches 77. Accordingly, the operation drive source 30 secures the rotating rollers 41 so as not to rotate, and does not move the wire 320 in the longitudinal axis direction X. Thus, unintended movement of the wire 320 and the indwelling unit 310 can be prevented. In particular, it is possible to prevent thrombi from accumulating in the indwelling unit 310, and the wire 320 from moving in the distal direction due to the blood flow in the distal direction without permission. It is also possible to appropriately maintain the shape of the indwelling unit 310 that is collecting thrombi, and prevent the thrombi from flowing beyond the indwelling unit 310.

As described above, the operation device 10 according to the second embodiment is the operation device 10 to which the proximal portion of the elongated treatment device 100 having the lumen 111 through which the wire 320 extending from the indwelling unit 310 indwelling in a living body lumen to the outside of the body passes can be joined, and includes: the housing 20 including the joining portion 21 to which the proximal portion of the treatment device 100 can be joined; the action unit 40 that is disposed on the proximal side of the joining portion 21, and is brought into contact with the wire 320 extending from the proximal portion of the treatment device 100, to apply, to the wire 320, the moving force for the wire 320 to move in the longitudinal axis direction X; the action unit drive source 75 that drives the action unit 40; and the operation switches 77 that are disposed so as to be operable from outside the housing 20, and start and stop the action unit drive source 75. With this arrangement, the operation device 10 can move the wire 320 in the direction opposite to the movement of the housing 20, in accordance of operations of the operation switches 77. Thus, it is possible to prevent the wire 320 from moving with movement of the housing 20 and the indwelling unit 310 from unintentionally moving with respect to a living body lumen. Thus, when the treatment device 100 is used, the operation device 10 can maintain appropriate positions and/or shapes of the indwelling unit 310 joined to the wire 320 extending through the lumen 111 of the treatment device 100, and the wire 320.

### Third Embodiment

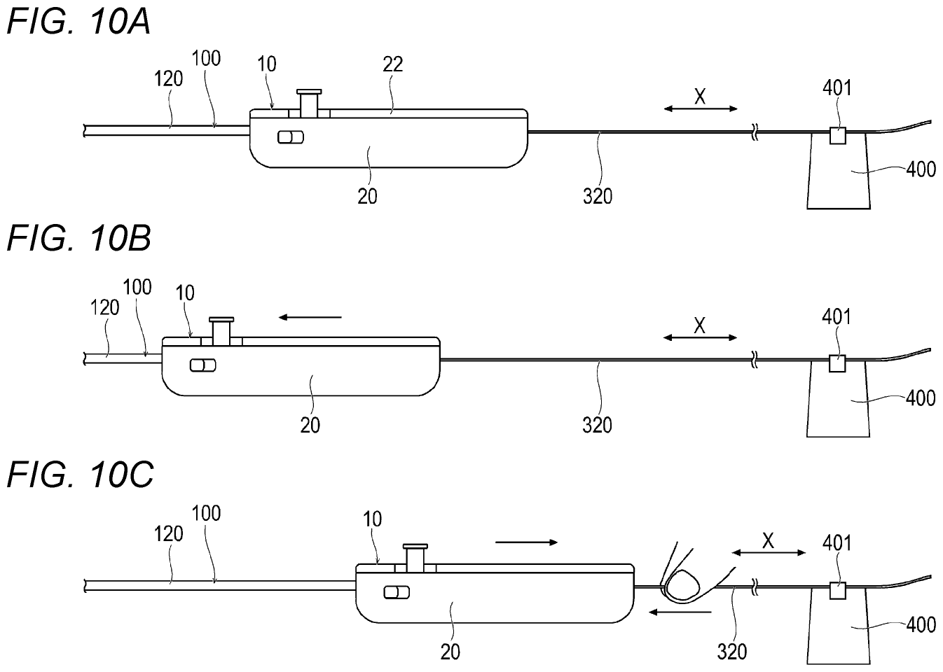

As illustrated in FIGS. 10A to 10C, an operation device 10 according to a third embodiment differs from that of the first embodiment in that an operator manually handles the wire 320. Note that components having the same functions as those of the first embodiment are denoted by the same reference numerals as those used in the first embodiment, and explanation of them is not made herein.

The operation device 10 does not include the action unit 40, the limiting mechanism 50, the movement detection unit 60, and the transmission unit 70. The operation device 10 includes the housing 20, the lid portion 22, the drive source 30 that drives the drive shaft 110, and the drive gear 31 that is rotated by the drive source 30.

Next, a method of use of a medical care system 1 including the operation device 10 according to the third embodiment is described.

As in the first embodiment, the operator first inserts the introducer sheath 200, the indwelling device 300, and the treatment device 100 into a blood vessel, and connects the operation device 10 to the treatment device 100. Accordingly, the wire 320 extending from the proximal end of the hub 160 of the treatment device 100 is placed at the proximal side of the operation device 10. Also, the drive gear 31 of the operation device 10 meshes with the driven gear 162 of the treatment device 100. Thus, the drive shaft 110 can be rotated by rotation of the drive source 30.

Next, it is preferable that the operator does not loosen the wire 320 extending from the proximal end of the hub 160 of the treatment device 100, between the proximal end of the treatment device 100 and a securing target object 400, which is the surgical table, an object placed on the surgical table, a drape, or the patient. The securing position of the securing target object 400 is preferably not largely separated from an extension line of the wire 320 extending from the proximal end of the hub 160 of the treatment device 100, and more preferably, is on the extension line.

Next, the operator actuates the drive source 30, to rotate the drive shaft 110 and the treatment unit 130. The operator then moves the operation device 10 alternately in the distal direction and the proximal direction, to crush a thrombus with the treatment unit 130 in the blood vessel.

As illustrated in FIG. 10B, when the operation device 10 is moved in the distal direction, the operator does not handle the wire 320 extending from the proximal end of the treatment device 100, and maintains a state of being secured to the securing target object 400. When the operation device 10 is moved in the distal direction, the wire 320 receives a force acting in the distal direction due to sliding with the treatment device 100 and the operation device 10, but does not move because the wire 320 is secured by the securing target object 400. With this arrangement, even when the operation device 10 moves, the wire 320 is kept from moving in the distal direction together with the operation device 10 by sliding with the treatment device 100 and the operation device 10. Therefore, the indwelling unit 310 secured to the distal portion of the wire 320 is less likely to be displaced, and the shape developed to cover the blood vessel lumen can be prevented from being canceled (or changed).

Further, as illustrated in FIG. 10C, when moving the operation device 10 in the proximal direction, the operator handles the wire 320 extending from the proximal end of the treatment device 100 with a finger, to move the wire 320 in the distal direction. When the operation device 10 is moved in the proximal direction, the wire 320 is pulled in the proximal direction by the sliding with the treatment device 100 and the operation device 10, and a slack occurs in the wire 320 between the proximal end of the treatment device 100 and the securing target object 400. However, the operator can move the wire 320 in the distal direction with respect to the operation device 10 so as to always eliminate the slack. As a result, even when the operation device 10 moves, the wire 320 is kept from moving in the proximal direction together with the operation device 10 by sliding with the treatment device 100 and the operation device 10. Therefore, the indwelling unit 310 secured to the distal portion of the wire 320 is less likely to be displaced, and the shape developed to cover the blood vessel lumen can be prevented from being canceled (or changed).

Furthermore, as illustrated in FIG. 10A, when the operation device 10 is held in a constant position, the operator does not handle the wire 320 extending from the proximal end of the treatment device 100, and maintains a state of being secured to the securing target object 400. In this manner, unintended movement of the wire 320 and the indwelling unit 310 can be prevented. In particular, it is possible to prevent thrombi from accumulating in the indwelling unit 310, and the wire 320 from moving in the distal direction due to the blood flow in the distal direction without permission. It is also possible to appropriately maintain the shape of the indwelling unit 310 that is collecting thrombi, and prevent the thrombi from flowing beyond the indwelling unit 310.

As described above, in the third embodiment, a method of operating the medical care system 1 is a method of operating the medical care system 1 that includes: the indwelling device 300 having the indwelling unit 310 indwelling in a living body lumen, and the wire 320 extending from the indwelling unit 310; the elongated treatment device 100 having the lumen 111 through which the wire 320 passes; and the operation device 10 that has the proximal portion of the treatment device 100 joined to the operation device 10 and operates the treatment device 100. The method includes: inserting the treatment device 100 into the living body lumen while the wire 320 extending from the indwelling unit 310 indwells in the living body lumen to the outside of the body is accommodated in the lumen 111; securing the wire 320 extending from the operation device 10 to a surgical table or the securing target object 400 that is an object placed on the surgical table; maintaining the securing of the wire 320 to the securing target object 400 when the operation device 10 is moved in the distal direction; and, when the operation device 10 is moved in the proximal direction, the wire 320 is moved in the distal direction with respect to the operation device 10. Accordingly, by the method of operating the medical care system 1, the wire 320 is secured to the securing target object 400, so that unintended movement of the indwelling unit 310 and the wire 320 due to a force received by the indwelling unit 310 from the blood flow or the like or a sliding force received by the wire 320 from the treatment device 100 or the like can be prevented. Thus, when the treatment device 100 is used, the method of operating the medical care system 1 can maintain appropriate positions and/or shapes of the indwelling unit 310 joined to the wire 320 extending through the lumen 111 of the treatment device 100, and the wire 320. When the operation device 10 is moved in the distal direction, the wire 320 is secured to the securing target object 400, so that it is possible to effectively prevent the wire 320 from moving in the distal direction together with the treatment device 100. Further, when the operation device 10 is moved in the proximal direction, it is difficult to prevent the wire 320 from moving in the proximal direction together with the treatment device 100, by securing the wire 320 to the securing target object 400. Therefore, the wire 320 is moved in the distal direction relative to the operation device 10, so that unintended movement of the indwelling unit 310 and the wire 320 can be prevented.

Note that, in the third embodiment, the operation device 10 may include the action unit 40 (a structure including two rotating rollers 41, for example) that grips the wire 320 extending from the proximal end of the hub 160 of the treatment device 100. At least part of the rotating rollers 41 is exposed to the outside of the housing 20 so that the operator can rotate the rotating rollers 41 with a finger. With such a configuration, the operator can move the wire 320 without slipping via the rotating rollers 41, instead of directly gripping and moving the wire 320.

Note that the present disclosure is not limited to the embodiments described above, and various modifications may be made by those skilled in the art within the technical idea of the present disclosure. For example, the treatment device joined to the operation device is not limited to a device that crushes thrombi. Therefore, the treatment device may be an atherectomy device for removing a lesion, or a diagnostic imaging catheter with the functions of intravascular ultrasound (IVUS), optical coherence tomography (OCT), or optical frequency domain imaging (OFDI). These treatment devices each include the rotating drive shaft 110.

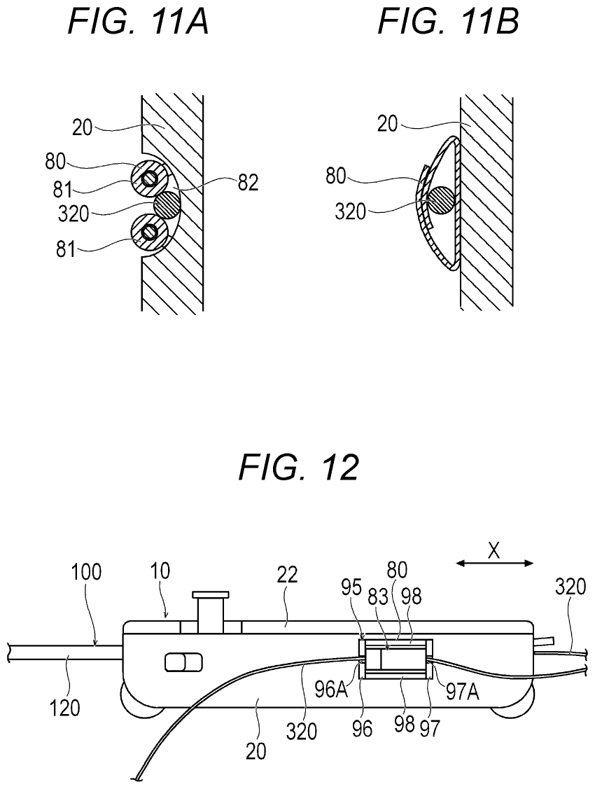

The form of the wire holding unit 80 in the first embodiment is not limited to any particular form. For example, as in a first modification illustrated in FIG. 11A, the wire holding unit 80 may include two holding rollers 81 rotatably held in the vicinity of the outer surface of the housing 20. The gap between the two holding rollers 81 is smaller than the outer diameter of the wire 320, and a holding space 82 capable of holding the wire 320 is formed between the two holding rollers 81 and the outer surface of the housing 20. The outer peripheral surface of each of the holding rollers 81 is preferably formed with a non-slippery material or structure. The outer surface of the housing 20 forming the holding space 82 is preferably slippery, and may be coated, for example, with a low-friction material. The low-friction material can be, for example, a fluororesin such as polytetrafluoroethylene. Since the outer surfaces of the two holding rollers 81 are less slippery, and the outer surface of the housing 20 is slippery, the wire 320 held in the holding space 82 is restricted from moving in the longitudinal axis direction X while being rotatable.

Also, the wire holding unit 80 may be a belt-like member that is formed with a hook-and-loop fastener, a magnet, or the like, and is capable of forming a ring that can be immediately secured and canceled, as in a second modification illustrated in FIG. 11B.

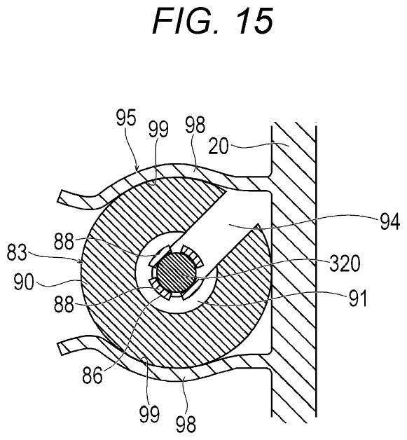

Also, the wire holding unit 80 may include a securing tool 83 secured to the wire 320, and a holding tool 95 that is disposed on the outer surface of the housing 20 and rotatably holds the securing tool 83, as in a third modification illustrated in FIGS. 12 to 16B. The securing tool 83 includes a securing tool main unit 84 and a cap 90. The securing tool main unit 84 is a tubular member in which a main-unit through-hole 85 is formed, and includes a diameter-reducible portion 86 formed on the distal side, and a male screw 87 formed on the outer peripheral surface on the proximal side of the diameter-reducible portion 86. The diameter-reducible portion 86 has a plurality of claw portions 88 arranged in the circumferential direction. Each of the claw portions 88 is a cantilevered portion in which the proximal side is a secured end, and the distal side is a free end. The distal side can be deformed so as to be bent in the radial direction of a wire holding tool 95. The plurality of claw portions 88 can clamp the wire 320 by bending so as to approach each other. In the securing tool main unit 84, a main-unit slit 89 is formed in the side wall from the distal end to the proximal end. The securing tool main unit 84 that is a tubular member has a discontinuous structure (a structure with less than 360 degrees) in the circumferential direction due to the main-unit slit 89.

The cap 90 is a tubular member in which a cap through-hole 91 is formed, and has, on the inner peripheral surface, a female screw 92 formed from the proximal side of the cap through-hole 91 and a guide portion 93 whose diameter decreases in the direction from the female screw 92 toward the distal side. A cap slit 94 is also formed in the side wall of the cap 90 from the distal end to the proximal end. The cap 90 that is a tubular member has a discontinuous structure (a structure with less than 360 degrees) in the circumferential direction due to the cap slit 94.

The holding tool 95 is a portion that is disposed on the outer surface of the housing 20, and rotatably holds the securing tool 83. The holding tool 95 includes a distal wall 96 on the distal side, a proximal wall 97 on the proximal side, and two holding walls 98 that rotatably hold the securing tool 83 between the distal wall 96 and the proximal wall 97.

The distal wall 96 slidably comes into contact with the distal surface of the securing tool 83, to restrict movement of the securing tool 83 in the distal direction. The distal wall 96 has a distal groove 96A that accommodates the wire 320. The proximal wall 97 slidably comes into contact with the proximal surface of the securing tool 83, to restrict movement of the securing tool 83 in the proximal direction. The proximal wall 97 has a proximal groove 97A that accommodates the wire 320.

Each of the holding walls 98 has a holding surface 99 that slidably sandwiches the securing tool 83. The holding surface 99 is formed in a concave shape so as to help prevent detachment of the securing tool 83 from the securing tool 83. The holding walls 98 can be elastically deformed to form a gap through which the securing tool 83 can pass, when the securing tool 83 is accommodated in the holding tool 95 and is removed from the holding tool 95.

The surfaces of the distal wall 96, the proximal wall 97, and the holding walls 98 that can come into contact with the securing tool 83 are preferably slippery, and may be coated, for example, with a low-friction material.

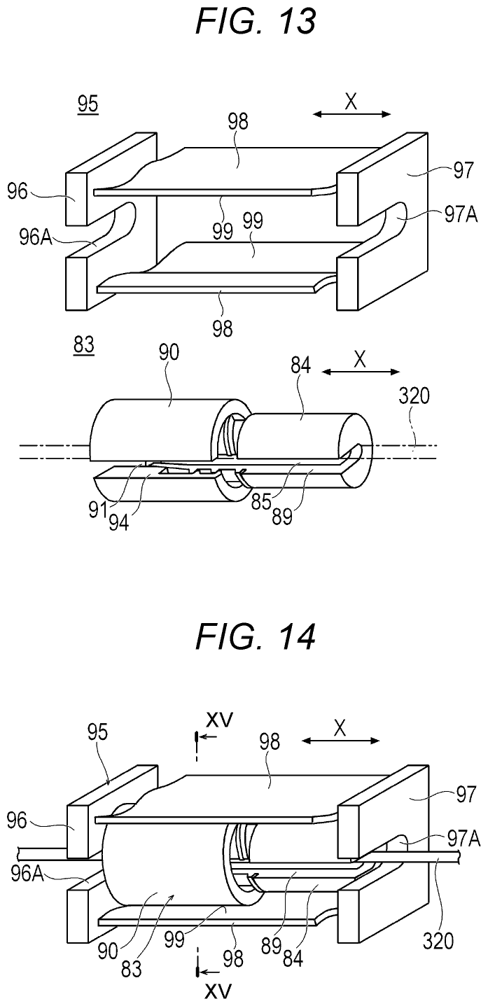

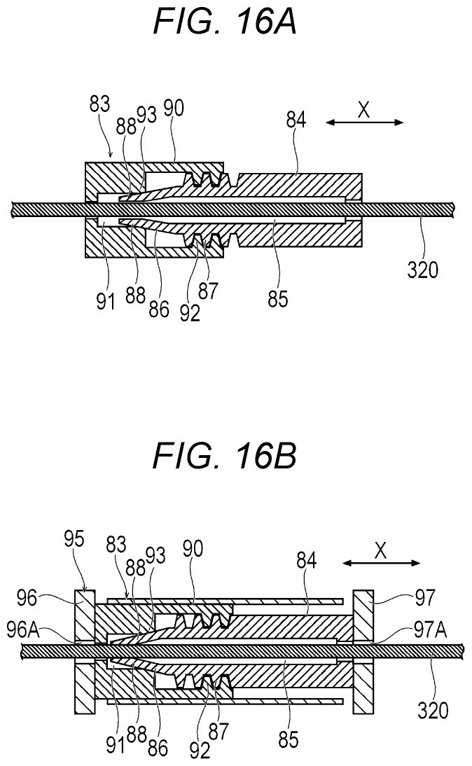

As illustrated in FIGS. 13 and 16A, the operator inserts the wire 320 into the cap slit 94 and the main-unit slit 89, while the cap 90 covers the securing tool main unit 84, and the cap slit 94 and the main-unit slit 89 match each other. As a result, the wire 320 is disposed in the cap through-hole 91 and the main-unit through-hole 85. Next, the operator rotates the cap 90 with respect to the securing tool main unit 84. As a result, as illustrated in FIG. 16B, the female screw 92 of the cap 90 and the male screw 87 of the securing tool main unit 84 are screwed together, and the diameter-reducible portion 86 of the securing tool main unit 84 comes into contact with the guide portion 93 whose diameter decreases in the direction toward the distal side at the back of the cap through-hole 91. Accordingly, the claws of the diameter-reducible portion 86 are guided by the guide portion 93 to bend inward in the radial direction, and the diameter of the diameter-reducible portion 86 becomes smaller. Thus, the plurality of claws of the diameter-reducible portion 86 firmly grips the wire 320, and the wire 320 is secured to the securing tool 83.

Next, as illustrated in FIGS. 12, 14, 15, and 16B, the operator inserts the wire 320 protruding in the distal direction from the securing tool 83 into the distal groove of the holding tool 95, and inserts the wire 320 protruding in the proximal direction from the securing tool 83 into the proximal groove 97A of the holding tool 95. Further, the operator pushes the holding walls 98 between the two holding walls 98 while bending the holding walls 98. Thus, movement of the securing tool 83 in the longitudinal axis direction X is restricted by the distal wall 96 and the proximal wall 97, while the securing tool 83 is rotatably held by the holding surfaces 99 of the two holding walls 98.

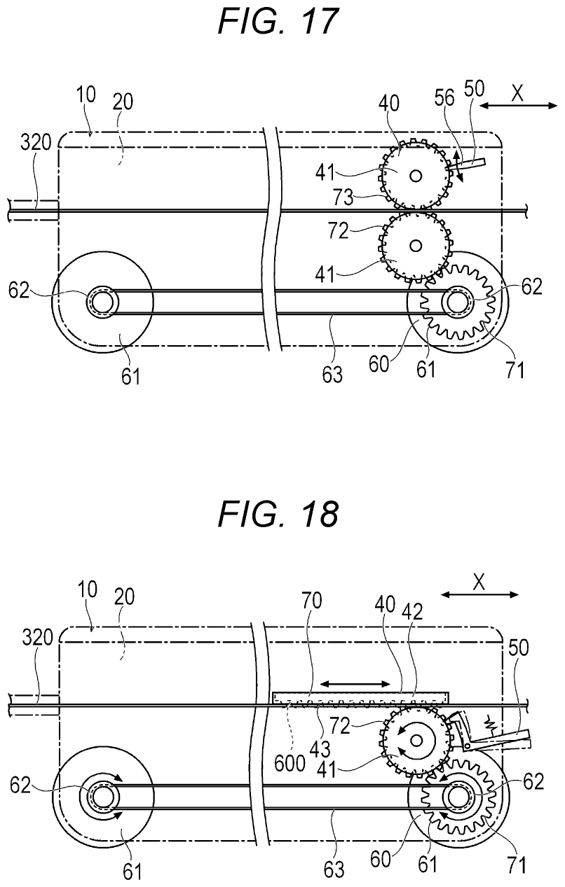

Further, the configuration of the limiting mechanism 50 in the first embodiment is not limited to any particular one, as long as movement of the wire 320 with respect to the housing 20 can be limited. For example, as in a fourth modification illustrated in FIG. 17, the limiting mechanism 50 may have an elastically deformable stopper 56. The stopper 56 has a cantilever-like shape, the secured end side is secured to the housing 20, and the free end side enters the tooth grooves of the third gear 73 (or the second gear 72). The stopper 56 restricts rotation of the third gear 73 with a predetermined holding force (elastic force). When receiving a force equal to or greater than the holding force, the stopper 56 is deformed, bent, and retracts from the tooth grooves, the rotating rollers 41 become rotatable, and the wire 320 in contact with the rotating rollers 41 becomes movable with respect to the housing 20. For example, the holding force of the stopper 56 is smaller than the force with which the operator moves the operation device 10, and is greater than the force received from the blood flow. Because of this, the limiting mechanism 50 can prevent thrombi from accumulating in the indwelling unit 310 serving as a filter, and prevent unintended movement of the wire 320 in the distal direction due to the blood flow in the distal direction. In a case where the operator wishes to move the operation device 10 without moving the indwelling device 300, the stopper 56 can be bent by a strong moving force, and the limiting mechanism 50 is automatically canceled. Thus, the operator can operate the operation device 10 without moving the indwelling device 300.

Further, as in a fifth modification illustrated in FIG. 18, the action unit 40 may include one rotating roller 41, and one linearly moving plate member 42 capable of holding the wire 320 between the rotating roller 41 and the outer peripheral surface of the one linearly moving plate member 42. The plate member 42 has a gripping surface 43 capable of gripping the wire 320 with the outer peripheral surface of the rotating roller 41. In the gripping surface 43, a groove may be formed in the longitudinal axis direction X so that the wire 320 can be stably held. The gripping surface 43 is formed with a non-slippery material. The gripping surface 43 is parallel to the longitudinal axis direction X and is movable in the longitudinal axis direction X. The transmission unit 70 includes a rack 600 that meshes with the second gear 72, and linearly moves in the longitudinal axis direction X. The rack 600 is secured to the plate member 42. The rack 600 has a plurality of teeth arranged in the longitudinal axis direction X. The rack 600 is held slidably in the longitudinal axis direction X by the housing 20. The gripping surface 43 is parallel to the longitudinal axis direction X and is movable in the longitudinal axis direction X.

When the detecting rollers 61 rotate to rotate the first gear 71, the second gear 72 meshing with the first gear 71 rotates, and the rack 600 meshing with the second gear 72 moves in the longitudinal axis direction X. As a result, the rotating roller 41 secured to the second gear 72 rotates, and the plate member 42 linearly moves. The peripheral speed of the outer peripheral surface of the rotating roller 41 at this point of time matches the moving speed of the rack 600. Thus, the rotating roller 41 and the plate member 42 can move the interposed wire 320 in the longitudinal axis direction X.

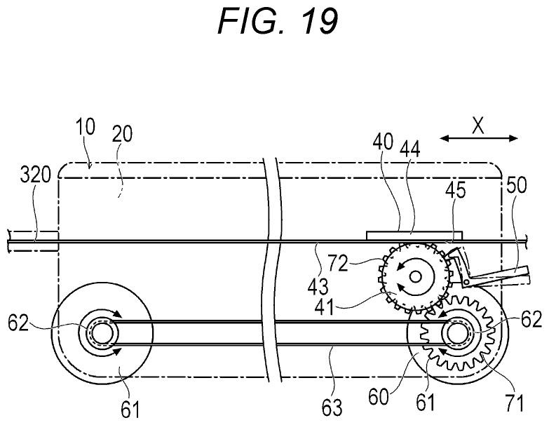

Further, as in a sixth modification illustrated in FIG. 19, the action unit 40 may include one rotating roller 41, and a securing plate 44 capable of holding the wire 320 between the rotating roller 41 and the outer peripheral surface of the securing plate 44. The securing plate 44 is secured to the housing 20, and has a sliding surface 45 capable of holding the wire 320 with the outer peripheral surface of the rotating roller 41. The sliding surface 45 is preferably slippery, and may be coated, for example, with a low-friction material. The sliding surface 45 is parallel to the longitudinal axis direction X. In the sliding surface 45, a groove may be formed in the longitudinal axis direction X so that the wire 320 can be stably held.

When the detecting rollers 61 rotate to rotate the first gear 71, the second gear 72 meshing with the first gear 71 rotates. As a result, the rotating roller 41 secured to the second gear 72 rotates. Thus, the rotating roller 41 can move the wire 320 interposed between the sliding surface 45 and the rotating roller 41 in the longitudinal axis direction X.

The detailed description above describes to an operation device for operating a treatment device inserted into a living body lumen, and a method of operating a medical care system. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An operation device configured to be joined to a proximal portion of an elongated treatment device, the elongated treatment device having a lumen through which a wire extending from an indwelling unit configured to be indwelled in a living body lumen, the operation device comprising:
a housing including a joining portion, the joining portion configured to be joined to the proximal portion of the treatment device; and
an action unit that is disposed on a proximal side of the joining portion, the action unit configured to be brought into contact with the wire extending from the proximal portion of the treatment device, and to apply, to the wire, a moving force for the wire to move in a longitudinal axis direction.

2. The operation device according to claim 1, wherein the action unit includes at least one rotating roller having an outer peripheral surface configured to contact with the wire.

3. The operation device according to claim 2, wherein at least part of the outer peripheral surface of the rotating roller is formed with a rubber material.

4. The operation device according to claim 2, further comprising:
a limiting mechanism configured to limit rotation of the rotating roller.

5. The operation device according to claim 1, further comprising:
a movement detection unit configured to detect a movement amount in the longitudinal axis direction of the housing and a movement direction of the housing.

6. The operation device according to claim 5, wherein the movement detection unit includes at least one detecting roller configured to rotate with respect to the housing, and at least part of an outer peripheral surface of the at least one detecting roller is located outside of the housing.

7. The operation device according to claim 5, further comprising:
a transmission unit configured to actuate the action unit to move the wire in a direction opposite to the movement direction by a distance corresponding to the movement amount detected by the movement detection unit.

8. The operation device according to claim 7, wherein the transmission unit includes at least one gear configured to transmit rotation detected by the movement detection unit to the action unit.

9. The operation device according to claim 1, further comprising:
a wire holding unit that is disposed outside the housing, and wherein the wire holding unit is configured to hold the wire and to rotate about the longitudinal axis direction while limiting movement of the wire in the longitudinal axis direction.

10. The operation device according to claim 1, further comprising:
the treatment device, the treatment device including a drive shaft that is configured to be rotatable about a longitudinal axis; and
the operation device further comprises a drive source configured to impart a rotational force to the drive shaft of the treatment member.

11. An operation device configured to be joined to a proximal portion of an elongated treatment device, the elongated treatment device having a lumen through which a wire extending from an indwelling unit configured to be indwelled in a living body lumen, the operation device comprising:
a housing including a joining portion, the joining portion configured to be joined to the proximal portion of the treatment device;
an action unit that is disposed on a proximal side of the joining portion, is the action unit configured to be brought into contact with the wire extending from a proximal portion of the treatment device, and to apply, to the wire, a moving force for the wire to move in a longitudinal axis direction;
an action unit drive source configured to drive the action unit; and
an operation switch configured to be operated from outside the housing, and to start and stop the action unit drive source.

12. The operation device according to claim 11, wherein the action unit includes at least one rotating roller having an outer peripheral surface configured to contact with the wire, and at least part of the outer peripheral surface of the rotating roller is formed with a rubber material.

13. The operation device according to claim 12, further comprising:
a limiting mechanism configured to limit rotation of the rotating roller.

14. The operation device according to claim 11, further comprising:
a movement detection unit configured to detect a movement amount in the longitudinal axis direction of the housing and a movement direction of the housing.

15. The operation device according to claim 14, wherein the movement detection unit includes at least one detecting roller configured to rotate with respect to the housing, and at least part of an outer peripheral surface of the at least one detecting roller is located outside of the housing.

16. The operation device according to claim 14, further comprising:
a transmission unit configured to actuate the action unit to move the wire in a direction opposite to the movement direction by a distance corresponding to the movement amount detected by the movement detection unit.

17. The operation device according to claim 16, wherein the transmission unit includes at least one gear configured to transmit rotation detected by the movement detection unit to the action unit.

18. The operation device according to claim 11, further comprising:
a wire holding unit that is disposed outside the housing, and wherein the wire holding unit is configured to hold the wire and to rotate about the longitudinal axis direction while limiting movement of the wire in the longitudinal axis direction.

19. The operation device according to claim 11, further comprising:
the treatment device, the treatment device including a drive shaft that is configured to be rotatable about a longitudinal axis; and
the operation device further comprises a drive source configured to impart a rotational force to the drive shaft of the treatment member.

20. A method of operating a medical care system that includes an indwelling device having an indwelling unit configured to be indwelled in a living body lumen, and a wire extending from the indwelling unit; an elongated treatment device having a lumen through which the wire passes, and an operation device that has a proximal portion of the treatment device, the operation device configured to be joined to the treatment device and to operate the treatment device, the method comprising:
inserting the treatment device into the living body lumen while the wire extending from the indwelling unit indwelling in the living body lumen to outside of the living body is accommodated in the lumen;
securing the wire extending from the operation device to a surgical table or a securing target object that is an object placed on the surgical table;
maintaining the securing of the wire to the surgical table or the securing target object when the operation device is moved in a distal direction; and
when the operation device is moved in a proximal direction, the wire is moved in the distal direction with respect to the operation device.
